(19) United States
(12) Patent Application Publication
(10) Pub. No.: US 2019/0321219 A1
(43) Pub. Date: Oct. 24, 2019

(54) RING IMPLANT

(71) Applicant: Implandata Ophthalmic Products GmbH, Hannover (DE)

(72) Inventors: Max Ostermeier, Seevetal (DE); Stefan Meyer, Hannover (DE); Burkhard Dick, Bochum (DE)

(73) Assignee: Implandata Ophthalmic Products GmbH, Hannover (DE)

(21) Appl. No.: 16/345,582

(22) PCT Filed: Oct. 26, 2017
(86) PCT No.: PCT/EP2017/077468

§ 371 Date: Apr. 26, 2019

(30) Foreign Application Priority Data
Oct. 28, 2016 (DE) 10 2016 221 371.7

(51) Int. Cl. A61F 9/00; A61B 3/16; A61B 5/00

(52) U.S. Cl. CPC A61F 9/0017; A61B 3/16; A61B 5/01; A61B 5/6821; A61B 5/6867; A61B 5/0031

(57) ABSTRACT

In order to make available an implant for implanting in an eye and/or on an eye, with fixing means arranged in a first plane and with a recess extending substantially in the first plane, which implant avoids the disadvantages of the prior art and can be better implanted, particularly as regards measurement of the intraocular pressure and reduced trauma to a patient, it is proposed that the implant, in a second plane at a distance from and substantially perpendicular to the first plane, has holding means for holding at least one sensor module having a sensor and/or at least one sensor module.

### CROSS-REFERENCE TO RELATED APPLICATIONS

This patent application is the US National Phase Under 371 of International Patent Application No. PCT/EP2017/077468, entitled “RING IMPLANT,” naming as inventors Max Ostermeier, Stefan Meyer, and Burkhard Dick, and filed Oct. 26, 2017, which application claims priority to German Patent Application No. 102016221371.7, filed Oct. 28, 2016, which patent documents are incorporated by reference herein in their entireties and for all purposes.

The present invention relates to an implant for implanting in an eye and/or on an eye having fixing means arranged in a first plane and having a recess extending substantially in the first plane.

Implants of this type are known from DE19945879A1 and DE102004061543B4. The known implants have a pressure sensor for measuring intraocular pressure and a telemetry coil for transmitting the measured data to a data reader. They are intended for implanting in the ciliary sulcus of the eye in a human patient, wherein a foldable plastics material ring containing the pressure sensor and the telemetry coil can be deployed so that the plastics material ring is fixed via the furrow bottom in the ciliary sulcus.

After implanting of the known implants, however, there is a potential risk that injury to the iris and increased intraocular pressure may occur in a patient due to the uveal contact.

Another disadvantage is that the eye has to be measured extensively before implanting one of the known implants, so that an implant suitable for fixing may be selected on the basis of the size of the ring diameter associated with the implants. Specifically, therefore, in known implants, there is the danger that they are fixed defectively.

The danger of a defective fixing associated with the known implants can have serious consequences for a patient. In the case of a defective fixing, migration and/or rotation of the known implant can occur after implanting, in that the implant and thus possibly the sensor fastened thereto, together with the telemetry unit, penetrate into the eye tissue and/or the iris. An increase in intraocular pressure, defects in the iris or shading of the optical axis of the eye are possible consequences.

The object of the present invention is therefore to provide an implant of the type mentioned at the outset which, by overcoming the abovementioned disadvantages of the prior art, may be implanted in an improved manner, in particular with regard to the measurement of intraocular pressure and the reduction of trauma to the patient.

According to the invention, this object is achieved in that a generic implant, in a second plane at a distance from and substantially perpendicular to the first plane, has holding means for holding at least one sensor module having a sensor and/or at least one sensor module. The implant according to the invention thus has, for example, an annular disc of thickness td having a circular opening of radius r for fixing the implant in a capsular bag, for example, and a rectangular mounting plate having a length l, width w and thickness t_{P} for mounting a pressure sensor with microelectronics and a coil for telemetric transmission of measurement data of the pressure sensor.

The fixing means and the holding means in the implant according to the invention in the case of the exemplary annular disc and mounting plate are positioned perpendicular to the radius r at a distance d>0 mm. In an advantageous manner, the implant according to the invention can be implanted in a human patient, for example, in such a way that the holding means are spatially separated from the fixing means along the optical axis of the eye and thus from the location of fixing. Advantageously, the risk of collision with the eye caused by the holding means or the sensor module is thus minimised. This is also advantageous with respect to an artificial lens to be implanted in the capsular bag, so that a mounting of the artificial lens on the implant according to the invention, which limits the selection of commercially available artificial lenses, is advantageously eliminated.

Since in the implant according to the invention the holding means or the sensor module are arranged substantially parallel to the fixing means and thus, for example in the case of a fixing in the capsular bag, parallel or nearly parallel to the iris of a patient, advantageously a mechanical contact of the implant according to the invention with the iris is avoided. The implant according to the invention can consequently minimise the risk of clinical or subclinical traumatic events on the iris, such as pigment abrasion, which typically increases the drainage resistance for the aqueous humour and causes an intraocular pressure increase and can cause what is known as church window syndrome (a syndrome in which light that shines through iris defects causes a visual glare effect).

An advantageous embodiment of the invention provides that, in the implanted state of the implant, the holding means and/or the sensor module can be arranged in a region between the capsular bag and the iris of the eye. Advantageously for the long-term monitoring of one or more clinical parameters, such as intraocular pressure in a human patient, according to this embodiment of the implant according to the invention, the sensor module is mostly protected against mechanical influences such as capsular bag shrinkage, cataract formation and capsular bag fibrosis.

Moreover, in contrast to the prior art, it is advantageously possible for the implant according to the invention to provide if necessary for a removal or replacement of the sensor module by a comparatively simple ophthalmological surgical procedure in human patients, for example.

Another advantage of the implant according to the invention is that due to the spatial separation between the fixing means on the tissue provided for implantation, for example, the capsular bag and the holding means or the sensor module in the spatial region between the capsular bag and the iris, the measuring accuracy of a pressure sensor mounted on the holding means, for example, is not affected by patient-specific anatomical conditions in the eye, which are usually caused by post-operative healing processes and body reactions.

In a further advantageous embodiment of the invention, the implant according to the invention has at least one spacer element between the holding means and the fixing means. The spacer element according to the present embodiment of the implant according to the invention may, for example, be a wire-shaped holding arm, one end of which is attached to the fixing means, while the other end is attached to the holding means or the pressure sensor. The implant according to the invention thus advantageously ensures the preferred spatial separation, which ensures a non-traumatic implant and also extends the selection range of commercially available artificial lenses, between the fixing means, i.e. an open annular disc in a ring segment, and the holding means or the sensor module, thus between the fixing point and the location of the holding means or of the sensor module.

The described embodiment can be further improved in that the spacer element has a portion which can be arranged on an edge surrounding a recess of a capsular bag. The spacer element of the implant according to the invention can therefore, for example, be provided with an undulating portion via which the spacer element is led, in a non-traumatic way, out of the opening of the capsular bag created during cataract surgery. This means that the portion is shaped according to the anatomical conditions of the opening of the capsular bag. In this way, in the implant according to the invention, it is ensured that the holding means or the sensor module may be arranged outside of the capsular bag, in the implanted state, which is advantageous, for example, for intraocular pressure measurement and the implanting of commercially available lenses.

The described improved embodiment of the invention can be further improved if the portion is directed radially outwards towards a radius of curvature defined locally at the edge. The spacer element of the implant according to the invention is thus bent, for example, in a portion in a hook-shaped manner such that the one end of the holding arm projects, for example, into the space between the anterior capsular bag and the posterior iris. This reduces the overall dimensions of the implant according to the invention in the direction parallel to the optical axis of the eye, whereby the implant according to the invention can be implanted in surgical practice relatively easily and in small surgical openings as are usual for an eye surgeon, and thus in a less traumatic way.

In addition, when the holding means or the sensor module are mounted on the hook-like bent portion, for example, a shading of the optical axis of the eye, which negatively affects the eyesight, is avoided, since the holding means according to this embodiment are positioned, in the implanted state, with a relatively large spacing in the direction transverse to the optical axis of the eye.

In a further advantageous embodiment of the invention, it is proposed that the fixing means and the holding means and/or the sensor module are mountable on one another in a mechanically reversible way. Advantageously, with regard to a trauma-free implanting as well as the interaction with a multiplicity of commercially available artificial lenses, the implant of the invention according to this embodiment, provides that the fixing means, such as an annular disc fixed in a capsular bag, and the holding means, such as a mounting plate having punched holes, or the sensor module, which comprises, for example, a pressure sensor with microelectronics and an inductive coil for transmitting data measured by the pressure sensor to a receiver, are mounted intraocularly on one another after implanting by means of a latching arrangement, in order to be able to separately inject or implant elements of the implant according to the invention. Advantageously, the implant according to the invention thus allows smaller surgical incisions for access to the anterior chamber of the eye in a human patient.

In a particularly preferred embodiment of the invention, it is proposed that the fixing means are designed as a capsular tension ring. If the fixing means of the implant according to the invention are designed as a capsular tension ring, such as a plastics material ring of almost 360°, it is possible that the implant according to the invention is advantageously implanted in the capsular bag in a human patient. This is due to the fact that the capsular bag is typically the least risky implant position in the anterior chamber of the eye, so that the risk of a measurement deviation mechanically induced by fibrosing or capsular bag shrinkage with respect, for example, to a measurement of the intraocular pressure, is advantageously eliminated.

A further advantage of the implant according to the invention is that the fixing means designed as a capsular tension ring can usually be implanted in the capsular bag virtually without trauma, without any further constructional measures, together with an artificial lens. Thus, the implant according to the present invention extends the spectrum of artificial lenses to a multiplicity of commercially available or special optics-containing artificial lenses that may be implanted in a human in the case of medically preferred monitoring of clinical parameters following cataract surgery. The inventive implant according to the present embodiment may be advantageously implanted easily and also safely in particular using a small-scale cutting technique by means of a simple or standard injector for artificial lenses or capsular tension rings by an implant technician (clear cornea <3.5 mm or preferably <3.0 mm or <2.8 mm or 2.5 mm). Since in the implanted state of the implant according to the invention due to fixing means formed by a capsular tension ring, after implanting in the capsular bag, also contact with perfused tissue structures, which also tend to scarring or dialysis, is also minimised, the implant according to the invention is in particular more gentle on the eye of a human patient affected by the implant.

According to a further aspect of the present invention, the fixing means have at least one support element which can be supported by the tissue structure in the direction transverse to the optical axis of the eye. In an advantageous manner, this ensures that the implant according to the invention may be fixed transversely to the optical axis of the eye, for example non-positively by means of, for example, a biasing force and/or by means of at least one form-fit between the support element and the tissue structure. A ‘tissue structure’ refers to any structure that is suitable for fixing the inventive implant with respect to the optical axis of the eye through the fixing means, which may be provided, for example, by a capsular tension ring. This means in particular the tissue structure of a pseudophakic capsular bag, so that the support element, which may have the form of an arcuate portion of a capsular tension ring, for example, is supported on the inner side of the capsular bag. In this way, migration and/or rotation of the implant according to the invention is advantageously avoided since the implant according to the invention can be fixed with respect to the optical axis of the eye and in particular transversely to the optical axis of the eye.

For example, a pressure sensor for measuring the intraocular pressure and/or a sensor for measuring the glucose level and/or a temperature sensor for temperature measurement and/or an optical sensor and/or an acoustic sensor and/or an optical micro camera and/or a spectrometer and/or microprocessors and/or photovoltaic elements and/or energy stores and/or data storage means may be mounted on the holding means, for example, by means of an adhesive attachment. The holding means may be a frame, preferably a closed or C-shaped frame, on which, for example, the pressure sensor cast into a polymer matrix is attached, with a form-fit via a tongue-and-groove connection, thus facilitating the mounting of the pressure sensor to the frame.

The frame may also be cast into the polymer matrix together with the pressure sensor, which is advantageous for the assembly. The transmission of the data measured by the sensor can occur in this case via an induction coil of a telemetry unit to an external receiver, which is located outside the eye. The telemetry unit can advantageously be cast preferably together with the pressure sensor and the frame, into the polymer matrix, which is advantageous for a compact construction of the inventive implant. The implant according to the invention may be advantageously used with the holding means for monitoring clinical parameters of the eye, such as the intraocular pressure.

In a preferred embodiment of the implant according to the invention, the fixing means comprise at least one deflecting element for deflecting a support element in a deflection plane, wherein the deflection plane is positioned in the implanted state perpendicular to the optical axis of the eye. Advantageously, by means of the deflecting element, which can be configured, for example, as an elastic spring, a deflection of the support element transversely to the optical axis of the eye is achieved, so that the fixing of the implant according to the invention, obtained by the support element, i.e. the support of an outer peripheral portion of a capsular tension ring on the inner side of a capsular bag, for example, covers the naturally occurring diameter range of the capsular bag. In other words, by means of the deflecting element, the implant according to the invention is suitable, for example, both for fixing in patients with a relatively small capsular bag and in patients with a relatively large capsular bag. Advantageously, therefore, the storage of differently dimensioned implants is eliminated, which reduces the logistical effort and associated costs both for the eye clinic and for the manufacturer and leads to a reduction in sources of error.

Since the deflection plane in the implant according to the invention is also positioned perpendicular to the optical axis of the eye, in the implant according to the invention advantageously the support of the support element and thus the occurrence of torques negatively influencing the fixing is minimised, whereby the implant according to the invention is advantageously mainly subject to reaction forces in the deflection plane.

Another preferred embodiment of the implant according to the invention provides that the deflecting element is able to recover its shape after deformation of the deflecting element, wherein the deflection is based on the shape recoverability. Due to the shape recoverability, i.e. the property of the deflecting element of having a dynamic shape change which tends towards a non-deformed state, such as in the case of the relief of a prestressed elastic spring, the deflecting element, such as a closed or open capsular tension ring of a flexible material, may unfold, advantageously automatically, i.e. without any or almost no intervention by a surgeon performing the implanting, for example, so that the support element, such as an outer peripheral portion of a capsular tension ring, moves transverse to the direction of the optical axis, for example, towards the inside of the capsular bag, until a contact between the support element and the capsular bag occurs.

Due to the shape recoverability of the deflecting element, it is also advantageously possible for the implant according to the invention to be implanted particularly easily and safely according to a small-scale cutting technique using a relatively simple or standard injector for intraocular lenses (parameters of the clear corneal technique, <3.5 millimetres or preferably <3 millimetres or more preferably <2.8 millimetres or <2.5 millimetres). The advantages associated with the implant according to the invention are also expressed by the fact that the shape recoverability of the deflecting element, such as the deflection of a deformed spring consisting of a polymer having shape memory properties after a certain switching temperature has been exceeded, allows for a nearly universal adaptation of the support element, such as the bow-shaped portions of a spring, to the anatomical characteristics of the eye, in particular the diameter spectrum of the capsular bag.

Advantageously, with regard to the fixing of the implant according to the invention, preoperative dimensioning is therefore eliminated. This is due to the fact that even clinics with modern facilities often do not have the measuring apparatuses required for an accurate measurement of the anatomical conditions of the eye, such as the diameter of the capsular bag. In addition, methods for estimating the anatomical characteristics on the basis of biometric data are often subject to errors and thus generally unsuitable. Accordingly, the implanting of the implant according to the invention is advantageously associated only with a low operative, in particular preoperative, effort.

Preferably, in the implant according to the invention, it is provided that the shape recoverability is elastic. Consequently, the deflecting element can be composed of a mechanical annular spring, for example. This means that, advantageously for the fixing of the implant according to the invention, an elastic biasing force in the capsular bag caused by the spring ensures a local abutment of the support element, i.e. for example the portion of an annular spring having a relatively large radius of curvature, transverse to the optical axis of the eye.

An implant according to the invention may also be designed so that the shape recoverability can be stimulated via a stimulus. The shape recoverability can therefore be switchable, which is advantageous for implanting the implant according to the invention. A ‘stimulus’ therefore refers to any external stimulus that causes a shape memory effect in a suitable material. Consequently, the deflecting element of the implant according to the invention can advantageously be implanted in a deformed state in such a way that after a physician performing the implanting has found a suitable position for fixing the implant according to the invention, such as the capsular bag, the deflecting element unfolds in a way which is advantageous for fixing due to a shape memory effect caused by a stimulus, such as a UV light pulse.

In a further development of the invention, the stimulus is a temperature change. This is a stimulus which is particularly simple and thus particularly favourable for implanting, in view of minimising the operating effort, for example, through which optionally a shape memory effect can be caused in metals and polymers. For example, a spring made of shape memory polymer may serve as a deflecting element in the implant according to the invention.

If the shape memory polymer is heated above a composition-specific temperature, a shape recovery of the spring takes place, i.e. a deflection of the exemplary deflecting element, such that its support element, for example, is supported on the inside of the capsular bag. Obviously, also the shape memory effect known from metals is advantageously applicable in that the deflecting element of the implant according to the invention consists of such a metal.

The deflecting element and/or the support element preferably consists of a polymer, in particular a biocompatible polymer. This is advantageous in view of a possible trauma-free fixing of the implant according to the invention, since a multiplicity of polymers have a rigidity suitable for the implanted state, that is to say a modulus of elasticity which confers to the deflecting element, that is to say, for example, to a capsular tension ring, and/or to the support element, that is to say an outer peripheral portion of the exemplary capsular tension ring, a dimensional stability and at the same time an elastic yielding, which does not injure the eye tissue

If, in the case of the implant according to the invention, the deflecting element and/or the support element consists, at least in portions, of a material having shape memory properties, the deflecting element unfolds in a particularly advantageous manner. This is because the implant according to the invention may be correspondingly conveniently introduced in a tube-like injector, for example, which is advantageous for the implanting of the inventive implant.

After exiting the injector at a position intended for implanting, such as the capsular bag, the deflecting element such as a polymeric capsular tension ring, unfolds due to a shape memory effect, in accordance with a shape intended for the fixing of the implant, such as a curved shape, so that the support element is supported on the inside of the capsular bag.

‘Shape memory properties’ or ‘memory effect’ refer to every material property of a metal or polymer, which, due to a phase change or a change in the chemical crosslinking of polymer chains, allows for a change in shape of the deflecting element on a macroscopic scale, in particular in the millimetre or centimetre order of magnitude, starting from a deformed state of the deflecting element.

According to a variant of the invention, the deflecting element is resistant to dents in a direction perpendicular to the deflection plane. This ensures, in view of a fixing of the inventive implant which is as non-traumatic as possible, that the deflecting element, i.e. a compression spring, for example, the elastic portions of which are in the deflection plane, for example, do not bulge out in the implanted state along the optical axis, which would possibly otherwise lead to injuries and long-term trauma of the iris and other surrounding tissues.

In a further advantageous embodiment of the implant according to the invention, it is provided that the deflecting element and/or the support element are arc-shaped at least in part. This has a positive effect on the shape recoverability of the deflecting element which is preferred for implanting the implant according to the invention or the elastic yielding of the support element required for injury-free implanting, such as the arcuate portion of a compression spring made of a shape-memory polymer. In this case, deflecting elements and/or support elements with C- and/or Z-shaped portions lying in part in the deflection plane are particularly preferred.

Preferably, in the implant according to the invention in the implanted state, the holding means may be positioned at a distance from the optical axis of the eye in the direction transverse to the optical axis of the eye. Thus, advantageously, in the implant according to the invention, the holding means provided with a plate, for example, are arranged outside the optical axis of the eye, so that a shading of the optical axis of the eye impairing the sight of a patient is avoided.

Advantageously, in a further preferred embodiment of the implant according to the invention, the fixing means, such as a bending wire, are cast with the holding means, such as a frame, in a polymer matrix or are glued into corresponding openings.

Finally, in a further advantageous embodiment of the implant according to the invention, it is provided that the holding means are integrally formed with the sensor and/or the telemetry unit as a polymer casting. Accordingly, the implant according to the invention can advantageously be embedded in a silicone rubber matrix, for example, for minimally invasive implanting.

### BRIEF DESCRIPTION OF THE DRAWINGS

The invention is described by way of example in the following in a preferred embodiment with reference to the drawings, wherein further advantageous details are shown in the drawings.

Functionally identical parts are provided with the same reference numerals.

In detail, in the drawings:

FIG. 1 is a schematic sectional view of an eye, of a capsular tension ring implanted in the capsular bag of the eye according to the prior art and an artificial lens implanted in the capsular bag, to illustrate the embodiments of the implant according to the invention shown in the subsequent drawings;

FIG. 2 is a schematic plan view of an implant according to the present invention implanted in the pseudophakic eye according to FIG. 1 in accordance with a first embodiment having a sensor module;

FIG. 3 is a schematic plan view of an implant according to the present invention implanted in the pseudophakic eye according to FIG. 1 in accordance with a second embodiment without a sensor module;

FIG. 4 is a schematic sectional view of an implant according to the present invention, implanted in the pseudophakic eye of FIG. 1 according to a third embodiment without a sensor module; and

FIG. 5 is a schematic sectional view of an implant according to the present invention implanted in the pseudophakic eye according to FIG. 1 according to the third embodiment of FIG. 4 having a sensor module;

FIG. 6 is a plan view in the direction along the optical axis of an eye on an implant implanted in an eye, according to a preferred embodiment of the invention;

FIG. 7 shows a section of the implant according to FIG. 6 along the line II of FIG. 6;

FIG. 8 is a perspective view of an implant according to the invention in a further preferred embodiment;

FIG. 9 is a plan view from above of the implant of FIG. 8;

FIG. 10 is a plan view from below of the implant of FIGS. 8 and 9;

FIG. 11 is a lateral view of the implant of FIGS. 8 to 10 in the direction of arrow VI of FIG. 9;

FIG. 12a is a schematic representation of an implant according to the invention in a preferred embodiment;

FIG. 12b is a schematic representation of another implant according to the invention in a preferred embodiment;

FIG. 13 is a schematic plan view of an implant according to the invention in a further preferred embodiment;

FIG. 14 is a sectional view of the implant according to FIG. 8 along the line IX of FIG. 13;

FIG. 15 is a schematic plan view of an implant according to the invention in a further preferred embodiment;

FIG. 16 is a sectional view of the implant according to FIG. 10 along the line XI of FIG. 10;

FIG. 17 is a schematic plan view of an implant according to invention in a further preferred embodiment;

FIG. 18 is a sectional view of the implant according to FIG. 12 along the line XIII of FIG. 12;

FIG. 19 is a sectional view of an implant according to the invention in a further preferred embodiment;

FIG. 20 is a sectional view of an implant according to the invention in a further preferred embodiment; and

FIG. 21 is a schematic plan view of an implant according to the invention in a further preferred embodiment.

### DETAILED DESCRIPTION OF THE EMBODIMENTS

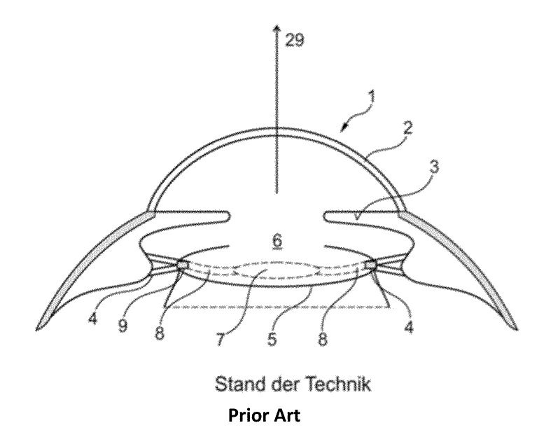

FIG. 1 is a schematic sectional view of an eye 1 with a cornea 2, an iris 3 and a capsular bag 5 attached to zonular fibres 4. In an opening 6, which has been opened in the course of cataract surgery, an artificial lens 7 is implanted in the eye 1, wherein the artificial lens 7 is fixed via haptics 8 to the capsular bag 5. In the capsular bag 5 a capsular tension ring 9 known from the prior art is fixed, which ring stabilises the capsular bag 5 in the direction transverse to an optical axis of the eye 29 by means of a biasing force.

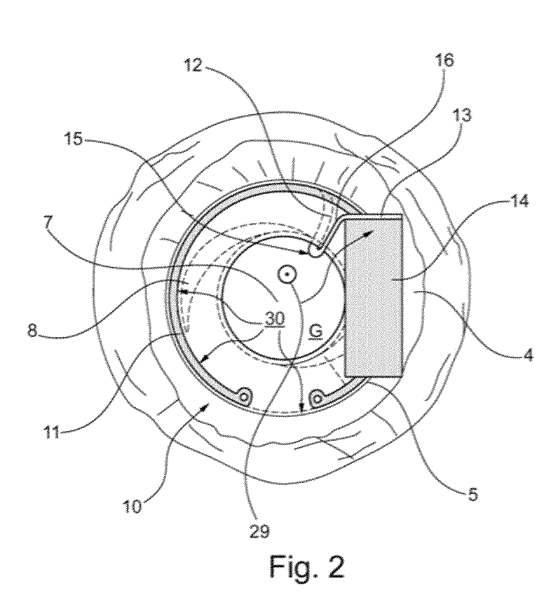

FIG. 2 shows, in a schematic plan view, an implant 10 according to the present invention in a first embodiment. The implant 10 is implanted in the eye shown schematically in FIG. 1 (the capsular tension ring 9 of FIG. 1 is replaced by the implant 10). The implant 10 has a capsular tension ring 11, which comprises a circular opening 30, via which the implant 10 according to the invention is fixed in the capsular bag 5 transversely to the optical axis 29, wherein the artificial lens 7 is fixed in the capsular bag 5 also by the haptics 8 transversely to the optical axis 29. The capsular tension ring 11 of the implant 10 according to the invention has been introduced into the capsular bag 5 through the opening 6 in the capsular bag 5. The capsular tension ring 11 of the implant 10 according to the present invention is analogous to a Cionni capsular tension ring (Cionni, R. J., Osher, R. H. (1995). Endocapsular ring approach to the subluxed cataractous lens. J-Cataract Refract-Surg. 21, 245-249) and has a holding arm 12. In the present case, the holding arm 12 is a holding arm of a Cionni capsular tension ring modified within the meaning of the invention.

The holding arm 12 of the capsular tension ring 11 of FIG. 2 has an attachment portion 13 on which a sensor module 14 is mounted. The sensor module 14 is composed of microelectronics elements (not shown) for digitising measurement data, an inductive coil for their telemetric transmission to a receiver and a pressure sensor for measuring intraocular pressure. However, the sensor module 14 is not limited to a pressure sensor, but may also provide a glucose sensor, a temperature sensor, an optical sensor, an acoustic sensor, a micro camera, or instead of the sensor module 13, a drug delivery device may be provided on the attachment portion 13. The holding arm 12 of the capsular tension ring 11 is led out of the opening 6 of the capsular bag according to FIG. 2 in the implant 10 according to the invention over an undulating portion 15.

The portion 15 of the holding arm 12 of the capsular tension ring 11 of FIG. 2 is integrally connected to a hook-shaped portion 16, which is substantially parallel to the plane defined by the capsular bag 5 and the zonular fibres 4. It can be seen from FIG. 2 that the implant according to the present invention is implanted together with the artificial lens 7 in the capsular bag 5, while the sensor module 13 and the attachment portion 13 are arranged outside the capsular bag 5 in order to minimise any possible risk of collision between the sensor module 14 and the artificial lens 7. It should also be noted that the artificial lens 7 may be formed or configured in various ways, without any adaptation to the implant according to the invention, such as the implant 10 in FIG. 2, being required.

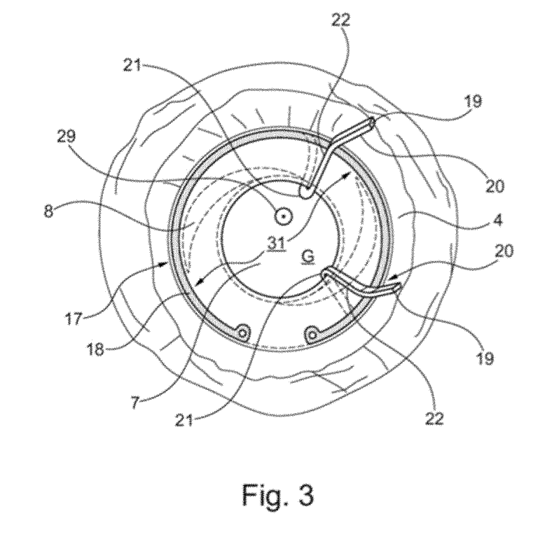

FIG. 3 is a schematic plan view of an implant 17 according to the invention in a second embodiment, which is implanted in an eye according to FIG. 1 such that the implant 17 is fixed in the capsular bag 5 in the presence of an artificial lens 7 via a capsular tension ring 18, which comprises a circular opening 31.

In FIG. 3, the capsular tension ring 18 of the implant 17 according to the invention is a Cionni capsular tension ring (Cionni, R. J., Osher, R. H. (1995). Endocapsular ring approach to the subluxed cataractous lens. J Cataract Refract—Surg. 21, 245-249) modified within the meaning of the invention, having two holding arms 19 according to the present embodiment. Portions 21 of the holding arms 19 of the implant 17 are undulating in such a way that they protrude out of the opening 6 of the capsular bag 5 and are provided, in a second plane spatially separated from a plane defined by the capsular tension ring 18, the normal of which extends parallel to the optical axis 29 of the eye, with an attachment portion 20 having a hook-shaped portion 22 extending parallel to the capsular bag 5 and the zonular fibres 4. On the two holding arms 19 on the attachment portion 22 a pressure sensor, a device for wireless data transmission and microelectronics may be mounted.

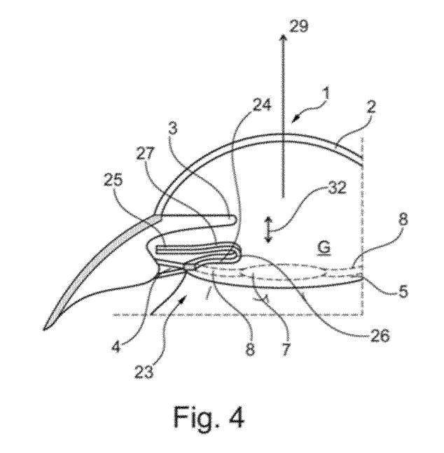

FIG. 4 shows, in a schematic sectional illustration, an implant 23 according to the invention in a third embodiment which, according to FIGS. 2 and 3, is implanted in an eye according to FIG. 1, such that a capsular tension ring (not shown) fixes the implant 23 in the capsular bag 5 transversely to the optical axis 29 of the eye. Similarly to implants of the invention from FIGS. 2 and 3, the implant 23 has at least one holding arm 24 which protrudes from an opening 6 in the capsular bag 5 via an undulating portion 26, so that a hook-shaped portion 27 substantially parallel to the plane defined by the artificial lens 7 is arranged in parallel outside the capsular bag, so that a plate for mounting a sensor module is mountable on an attachment portion 25.

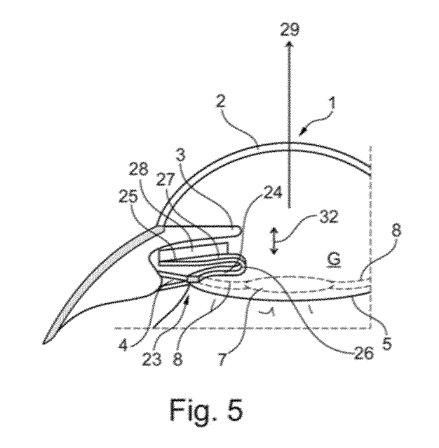

FIG. 5 shows the implant 23 of FIG. 4 with a sensor module 28, which is attached to the attachment portion 25, for example, by means of an adhesive connection (not shown in detail). FIG. 5 illustrates the spatial separation between the artificial lens 7, which is fixed, together with a capsular tension ring (not shown) of the implant 23 according to the invention in the capsular bag 5, along the optical axis 29 and the sensor module 28, so that the implant 23 according to the invention is arranged in a first plane and the sensor module 28 is arranged in a second plane such that, advantageously, any risk of collision between the implant 28 according to the invention and the artificial lens 27 due to a possible capsular bag shrinkage is minimised.

According to FIG. 5, since the implant 23 according to the invention is fixed in the capsular bag and the attachment portion 28 is disposed outside the capsular bag 5, namely in a region between the capsular bag 5 and the iris 3, the implant 23 according to the invention, and the implants according to the other embodiments of FIGS. 2 and 3, may be combined with a multiplicity of commercial artificial lenses as well as artificial lenses, which have refractive or multifocal optics, without the artificial lenses needing any constructive adaptation to the implants of the invention.

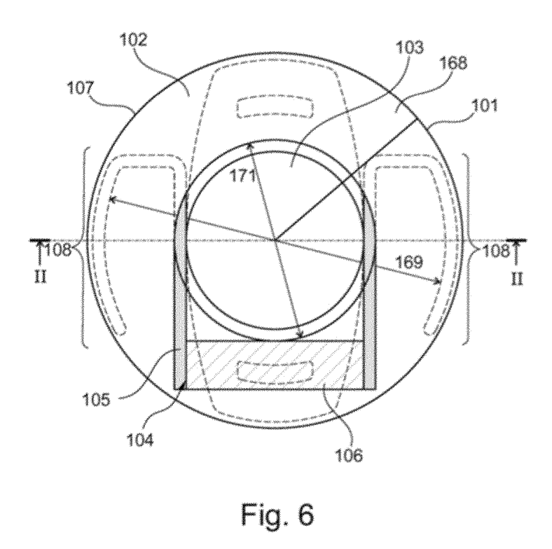

FIG. 6 shows, in the direction along the optical axis 101 of an eye 102, a plan view of the eye 102 with an artificial lens 103 and an implanted implant 104 according to a preferred embodiment of the invention. The implant 104 consists of two bent compression springs 105 and a plate 106 integrally formed with the compression springs 105, which is connected via an adhesive connection (not shown) to a sensor telemetry module (also not shown) for measuring intraocular pressure and for transmission of the data related to intraocular pressure to an external receiver.

FIG. 6 shows that the compression springs 105 press the implant 104 against a furrow bottom 107 of the capsular bag 168, which is formed analogously to the capsular bag 5 of FIGS. 1 to 5, so that the implant 104 is fixed transversely to the direction of the optical axis 101 via a bow portion 108 to the furrow bottom 107 by means of a biasing force of the compression springs 105. In this case, the plate 106 is arranged outside the artificial lens 103 in the direction transverse to the optical axis 101, in order to avoid shading of the artificial lens 103 that adversely affects the eyesight, wherein the implant 104 has been implanted through the opening 171 in the capsular bag 168 in the course of capsulorhexis.

FIG. 6 also shows that the implant 104 has an opening 170 which lies in the plane defined by the bow portions 108.

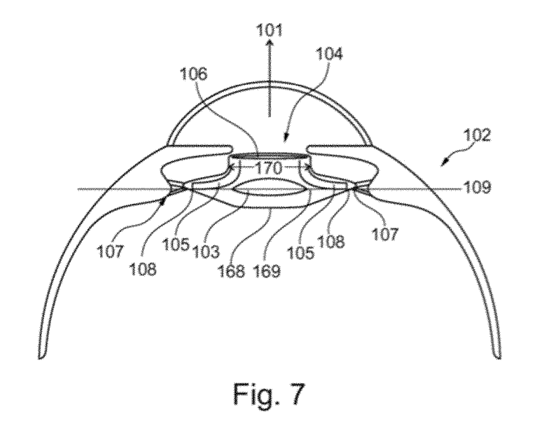

FIG. 7 is a sectional view of the implant 104 from FIG. 6. With reference to FIG. 7, it can be seen that the compression springs 105, when elastically deformed in a plane 109 which is perpendicular to the optical axis 101, recover their shape to such an extent that the bow portion 108 of the compression springs 105 is pressed against the furrow bottom 107 of the capsular bag 168, whereby the implant 104 is fixed transversely to the direction of the optical axis 101.

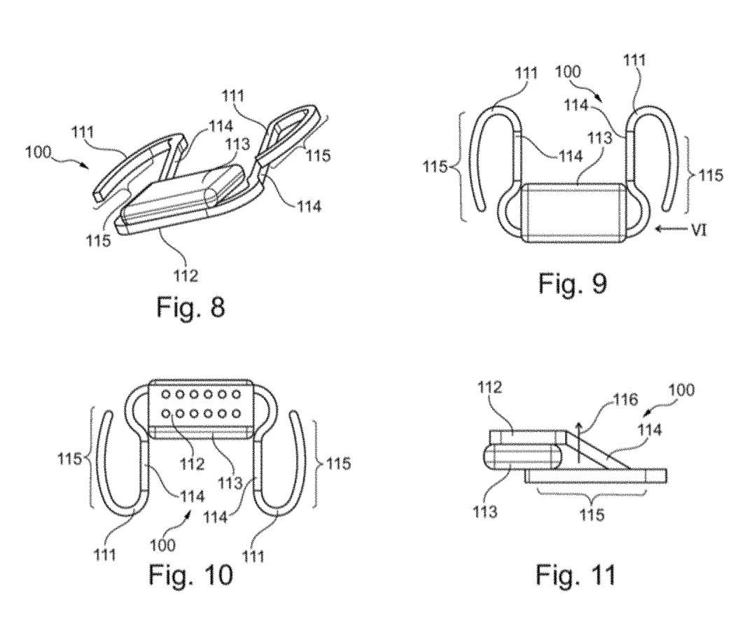

FIGS. 8 to 11 are different views of a further implant 100 according to the invention. Analogously to the implant 104 of FIGS. 6 and 7, the implant 100 according to FIGS. 8 to 11 consists of two compression springs 111 and a plate 112 formed integrally with the compression springs 111. The plate 112 is connected via an adhesive connection (not shown) to a sensor telemetry module 113.

An adhesive has flowed in the liquid state into the holes in the plate 112 shown in FIG. 10, so that the sensor telemetry module 112 is additionally anchored to the plate 112 by means of the adhesive, which has subsequently cured in the holes.

It is clear from the implant 100 shown in FIGS. 8 and 11 that the compression springs 111 are designed in an angulated manner in a middle portion 114 and that the bow portions are bent relative to the middle portion 114 by 180°. As a result, bow portions 115 of the compression springs 111 which may be supported by the furrow bottom of the capsular bag (not shown in FIGS. 8 to 11) are spatially separated from the plate 112 and thus from the sensor telemetry modules 113 along a direction 116 shown in FIG. 11. Through this spatial separation, due to the angled portion 114 of the compression springs 111 in the implanted state of the implant 100, it is ensured that the risk of collision of the plate 112 and/or the sensor telemetry module 113 with the iris of an eye is minimised. This is due to the fact that the implant 100 can be fixed via the bow portions 115 by means of an elastic shape recovery of the compression springs 11 in such a way that the bow portions 115 are arranged along the optical axis of the eye (cf. FIG. 7) closer to the iris than the plate 112 and the sensor telemetry module 113 of the implant 100.

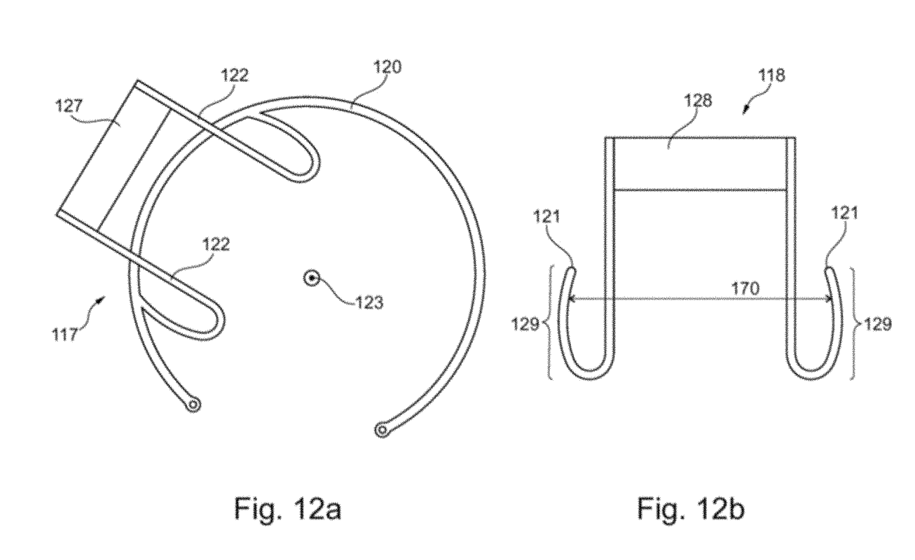

FIG. 12a schematically shows an implant 17 according to the invention in a further preferred embodiment. The implant 117 has a plate 127 which is connected to a Cioni-like capsular tension ring 120 via the holding arms 122. The plate 120 is spaced parallel to the direction vector 123 perpendicular to the capsular tension ring 120. The implant 117 is fixed in the furrow bottom (not shown) of a capsular bag (also not shown) via the capsular tension ring 120.

FIG. 12b schematically shows a further implant 118 according to the invention. The implant 118 consists, in addition to a plate 128, of two compression springs 121, which fix the implant without the help of a Cioni capsular tension ring in the furrow bottom of the capsular bag. Similarly to the implant 17 of FIG. 12a, the plate 128 of the implant 118 is set at a distance perpendicularly to a plane defined by the bow portions 129 of the compression springs 121. It can also be seen from FIG. 12 b that the implant 118 has an opening 170 which lies in the plane defined by the bow portions 129.

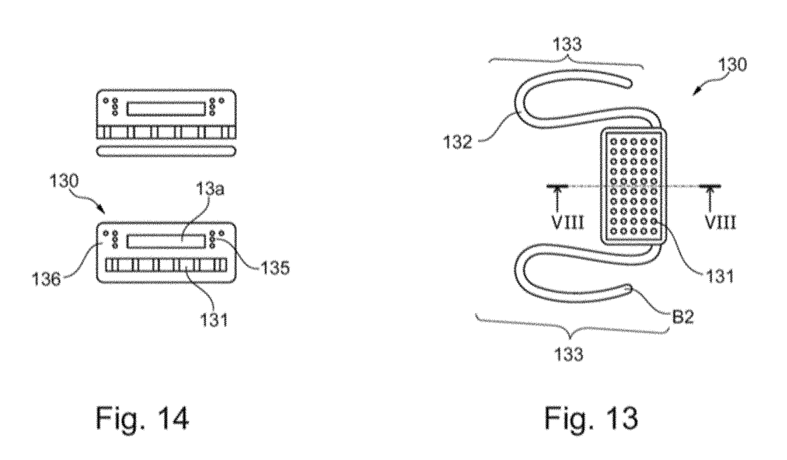

FIG. 13 shows an implant 130 in a further preferred embodiment according to the invention. The implant 130 consists of a perforated plate 131 and compression springs 132 having bow portions 133 provided for attachment to the furrow bottom of the capsular bag, which can be deflected by an elastic recovery of the compression springs 132 in the direction of the furrow bottom of the capsular bag.

FIG. 14 is a sectional view of the implant 130 of FIG. 13. The sectional view from FIG. 14 shows that, in the case of the implant 130, a pressure sensor 134 is surrounded, together with an induction coil 135, which telemetrically transmits measured data detected by the pressure sensor to a receiver, by a polymer matrix 136, so that the pressure sensor 134, the induction coil 135 and the plate 131 are present as a one-piece component in the implant 130. The polymer matrix 136 could flow in the liquid state into the holes in the plate 131, whereby the pressure sensor 134 and the induction coil 135 are anchored via the polymer matrix 136 in the holes in the plate 131. The polymer matrix 136 in this case consists of silicone rubber.

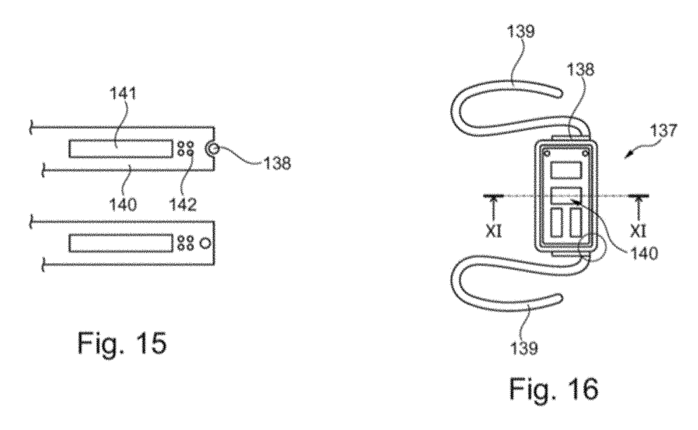

FIG. 15 is a schematic plan view of another implant 137 according to the invention. The implant 137 according to FIG. 15 is composed of compression springs 139 similar to the implants of FIGS. 5 to 14 and a frame 138 which is open on one side, in which, according to the sectional view of FIG. 16, a pressure sensor 141 embedded in a polymer matrix 140 of silicone rubber, is held together with an induction coil 142. The polymer matrix 140 was thus introduced into the frame 138 at the open side of the frame 138. The implant 137 is thus of modular construction, namely composed of a module consisting of the compression springs 139 and the frame 138 and a module, which is removable from the frame 118, consisting of the polymer matrix 40 made of silicone rubber comprising the pressure sensor 141 and the induction coil.

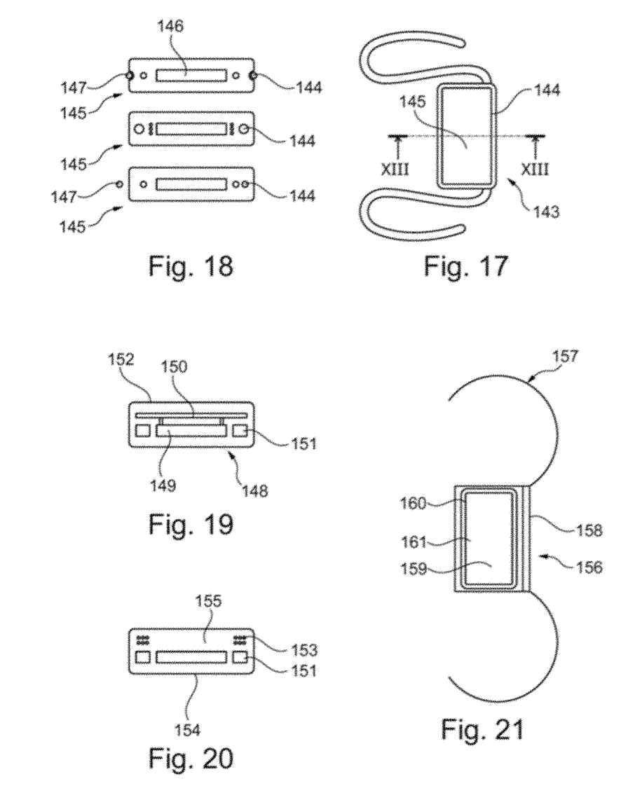

FIG. 17 is a schematic plan view of a further implant 243 according to the invention with a frame 244 which, in contrast to the frame 138 of the implant 137 according to FIGS. 15 and 16, is closed all around. In the frame 144 of the implant 143, similarly to the implant 137 of FIGS. 15 and 16, a polymer matrix 145 is supported, in which a pressure sensor 146 and an induction coil 147 are embedded. The sectional illustration of the implant 143 in FIG. 18 shows that the polymer matrix 145 is mounted in a form-fitting manner in the frame 144 of the implant 143 by means of a groove (FIG. 18, upper side), or that the frame 144 is completely embedded in the polymer matrix 142 (FIG. 18, centre) or that the frame 144 is embedded in part in the polymer matrix 145 (FIG. 18, lower side) by arranging a rail 147 of the frame outside the polymer matrix (FIG. 18, lower side).

FIG. 19 is a sectional view of an implant 148, in which a pressure sensor 149 and a planar coil 150 serving as a telemetry unit are embedded together with a holding frame 151 in a polymer matrix 152.

FIG. 20 is a sectional view of an implant 154 similar to the implant 48 of FIG. 19, but in which a coil 153 is embedded in a polymer matrix 155.

FIG. 21 is a plan view of an implant 156 according to the invention, in which a one-piece compression spring 157, which consists of a thread, is embedded at a portion 158 in a polymer matrix 159 together with a coil 160 and a pressure sensor 161. Thus, the implant 156 has a particularly compact construction, which is thus advantageous for implantation.

### LIST OF REFERENCE NUMERALS

1 eye
2 cornea
3 iris
4 zonular fibres
5 capsular bag
6 opening
7 artificial lens
8 holding arm
9 capsular tension ring
10 implant
11 capsular tension ring
12 holding arm
13 attachment portion
14 sensor module
15 portion
16 portion
17 implant
18 capsular tension ring
19 holding arm
20 attachment portion
21 portion
22 portion
23 implant
24 holding arm
25 attachment portion
26 portion
27 portion
28 sensor
29 optical axis
30 opening
31 opening
32 region
100 implant
101 optical axis
102 eye
103 lens
104 implant
105 compression spring
106 plate
107 furrow bottom
108 bow portion
109 plane
110 implant
111 compression spring
112 plate
113 sensor telemetry module
114 middle portion
115 bow portion
116 direction
117 implant
118 implant
120 capsular tension ring
121 compression spring
122 holding arms
123 holding arms
127 plate
128 plate
129 bow portion
130 implant
131 plate
132 compression springs
133 bow portion
134 pressure sensor
135 induction coil
136 polymer matrix
137 implant
138 frame
139 compression spring
140 polymer matrix
141 pressure sensor
142 induction coil
143 implant
144 frame
145 polymer matrix
146 pressure sensor
147 rail
148 implant
149 pressure sensor
150 planar coil
151 holding frame
152 polymer matrix
153 coil
154 implant
155 polymer matrix
156 implant
157 compression spring
158 portion
159 polymer matrix
160 coil
161 pressure sensor
168 capsular bag
169 opening
170 opening
171 opening of the capsular bag (capsulorhexis)

1. A method comprising:
implanting in and/or on an eye an implant with a fixing apparatus that is arranged in a first plane, having a recess extending substantially in the first plane, characterised in that the implant in a second plane at a distance from and substantially perpendicular to the first plane has a holding apparatus for holding at least one sensor module having a sensor and/or at least one sensor module.

2. The method of claim 1, characterised in that, in an implanted state of the implant, the holding apparatus and/or the sensor module is arranged in a region between a capsular bag and iris of the eye.

3. The method of claim 1, characterised in that the implant has at least one spacer element between the holding apparatus and the fixing apparatus.

4. The method of claim 3, characterised in that the spacer element has a portion which is arranged on an edge bounding a recess of a capsular bag.

5. The method of claim 4, characterised in that the portion is directed radially and outwardly in a direction of a radius of curvature locally defined on the edge.

6. The method of claim 1, characterised in that the fixing apparatus and the holding apparatus and/or the sensor module are mountable on one another in a mechanically reversible way.

7. The method of claim 1, characterised in that the fixing apparatus is formed as a capsular tension ring.

8. The method of claim 1, characterised in that the fixing apparatus has at least one support element, which is supported on a tissue structure in a direction transverse to an optical axis of the eye.

9. The method of claim 8, characterised in that the fixing apparatus has at least one deflecting element for deflecting the support element in a deflection plane wherein the deflection plane in the implanted state is positioned perpendicular to the optical axis of the eye.

10. The method of claim 9, characterised in that the at least one deflecting element is able to recover its shape after deformation of the at least one deflecting element, wherein the deflection is based on the shape recoverability.

11. The method of claim 10, characterised in that the shape recoverability is elastic.

12. The method of claim 10, characterised in that the shape recoverability is stimulated by a stimulus.

13. The method of claim 12, characterised in that the stimulus is a change in temperature.

14. The method of claim 9, characterised in that the at least one deflecting element and/or the support element comprises of a polymer, in particular a biocompatible polymer.

15. The method of claim 9, characterised in that the at least one deflecting element and/or the support element comprises at least in portions of a material having shape memory properties.

16. The method of claim 9, characterised in that the at least one deflecting element in the direction perpendicular to the deflection plane has a shape which is rigid with respect to dents.

17. A method comprising:
implanting in and/or on an eye an implant that is arranged in a first plane, having a recess extending substantially in the first plane;
holding at least one sensor module having a sensor and/or at least one sensor module in the implant in a second plane at a distance from and substantially perpendicular to the first plane; and
deflecting a support element in a deflection plane with at least one deflecting element, wherein the deflection plane is positioned in an implanted state perpendicular to an optical axis of the eye.

18. An implant comprising:
a fixing apparatus for implanting in and/or on an eye an implant;
a holding apparatus for holding at least one sensor module having a sensor in the implant; and
the sensor module, comprising the sensor,
wherein holding apparatus is spatially separated from the fixing apparatus along an optical axis of the eye.

19. The implant of claim 18, wherein the fixing apparatus has at least one support element, which is supported on a tissue structure in a direction transverse to the optical axis of the eye.

20. The implant of claim 19, wherein the fixing apparatus has at least one deflecting element for deflecting the support element in a deflection plane wherein the deflection plane in an implanted state is positioned perpendicular to the optical axis of the eye.
